(19) United States
(12) Patent Application Publication
(10) Pub. No.: US 2012/0143061 A1
(43) Pub. Date: Jun. 7, 2012

(54) TWO-DIMENSIONAL ARRAY ULTRASONIC PROBE

(75) Inventors: Yasuharu Hosono, Yokohama-shi (JP); Yohachi Yamashita, Yokohama-shi (JP); Kazuhiro Itsumi, Kawasaki-shi (JP)

(73) Assignee: KABUSHIKI KAISHA TOSHIBA, Tokyo (JP)

(21) Appl. No.: 13/300,024

(22) Filed: Nov. 18, 2011

Related U.S. Application Data
(63) Continuation of application No. 11/709,463, filed on Feb. 22, 2007, now abandoned.

(30) Foreign Application Priority Data
Jul. 12, 2006 (JP) 2006-192020

(51) Int. Cl. A61B 8/00

(52) U.S. Cl. 600/459

(57) ABSTRACT

A two-dimensional array ultrasonic probe includes a plurality of channels arranged apart from each other in a two-dimensional direction, each channel including a laminated piezoelectric element and an acoustic matching layer formed on the laminated piezoelectric element, the laminated piezoelectric element including a plurality of first and second electrodes arranged alternately within a piezoelectric body in a thickness direction of the piezoelectric body such that the side edges alone of the first electrodes and the second electrodes are exposed to the two mutually facing side surfaces of the piezoelectric body, respectively. The laminated piezoelectric element is mounted to a backing member. A signal side electrode and a ground electrode are formed to respectively extend from both side surface of the piezoelectric body to reach the backing member and are connected to the side edges of the first and second electrodes exposed to the side surface of piezoelectric body, respectively.

### CROSS-REFERENCE TO RELATED APPLICATIONS

This application is a continuation of U.S. application Ser. No. 11/709,463 filed Feb. 22, 2007, now pending, which is based upon and claims the benefit of priority from prior Japanese Patent Application No. 2006-192020, filed Jul. 12, 2006, the entire contents of both of which are incorporated herein by reference.

### BACKGROUND OF THE INVENTION

1. Field of the Invention

The present invention relates to a two-dimensional array ultrasonic probe, particularly, to a two-dimensional array ultrasonic probe prepared by arranging a plurality of channels including piezoelectric elements into a matrix form, which are used in an ultrasonic diagnostic apparatus and an ultrasonic defect detecting apparatus.

2. Description of the Related Art

In an ultrasonic probe, an ultrasonic wave generated by a piezoelectric element is radiated toward an object so as to have the object irradiated with the ultrasonic wave, and a reflected wave coming from an interface of the object differing in the acoustic impedance is received so as to make it possible to form an image showing the inner state of the object. An ultrasonic diagnostic apparatus for examining the inner region of the human body and a defect detecting apparatus for detecting the defect inside the metal welded portion are well known as ultrasonic imaging apparatus having the ultrasonic probe.

Particularly, the ultrasonic diagnostic apparatus is advantageous over X-ray diagnostic apparatus in that the apparatus permits observing the inner state of the human body without giving an irradiated effect to the human body and, thus, is widely used as a medical diagnostic apparatus. An ultrasonic probe comprising a piezoelectric element including a piezoelectric ceramic material (piezoelectric body) is used as an ultrasonic transmitter-receiver in the ultrasonic diagnostic apparatus. It is possible for an electronic scanning ultrasonic probe comprising a large number of very small piezoelectric elements arranged therein to form a tomographic image showing the inner state of the human body for diagnosis.

In an electronic scanning type ultrasonic probe comprising a plurality of piezoelectric elements arranged in one dimensional direction, it is possible to set optionally the focal point in the arranging direction of the piezoelectric elements by using the respective delay times of transmitted or received signals and in depth direction by selecting appropriately the number of columns of piezoelectric elements arranged in the ultrasonic probe. However, in a direction perpendicular to the arranging direction of the piezoelectric elements in the ultrasonic wave transmitting-receiving plane, the focus alone can be adjusted by an acoustic lens for adjusting the focal point. It follows that it is difficult to change the focal point dynamically. It should also be noted that the scanning method of the ultrasonic beam is limited to be performed in the two-dimensional direction, i.e., within the same plane, since the piezoelectric elements are arranged in the one-dimensional direction.

In recent years, a vigorous research have been done to develop a system having a two-dimensional array ultrasonic probe in which piezoelectric elements are arranged to form a two-dimensional matrix. In this system, the focal point of the ultrasonic wave is dynamically focused in all the directions by utilizing the ultrasonic probe. Also, the ultrasonic beam is scanned at a high speed in a three-dimensional direction so as to collect and display the three-dimensional ultrasonic image information. In the two-dimensional array ultrasonic probe, the piezoelectric elements are arranged in general in a manner to form a matrix having m rows and n columns (m×n). In order to carry out sufficiently the three-dimensional dynamic focusing and the three-dimensional beam scanning, it is desirable for the piezoelectric elements to be arranged at a very small pitch not larger than about 400 μm such that the matrix comprises at least 30 rows and at least 30 columns of the piezoelectric elements. Particularly, where the two-dimensional array ultrasonic probe is used for observing the human heart, the size of the probe head is desired for not larger than about 20 mm square in order to permit the ultrasonic beam to be incident on the heart through the clearance between the two adjacent ribs. The two-dimensional array ultrasonic probe having the particular head includes at least 900 withdrawing wirings.

In order to improve the performance of the two-dimensional array ultrasonic probe noted above, it is important for the piezoelectric elements to be miniaturized so as to permit the piezoelectric elements to be arranged at a high density within a limited area. However, it is necessary for the area of each piezoelectric element included in the two-dimensional array ultrasonic probe to be diminished in view of the demand for the two-dimensional array ultrasonic probe noted above. It follows that the capacitance of each piezoelectric element is rendered markedly smaller than that of the one-directional array ultrasonic probe, with the result that the sensitivity of the two-dimensional array ultrasonic probe is lowered.

Under the circumstances, it is known in the art to use a laminated piezoelectric element, which is constructed such that a plurality of electrodes are alternately arranged in the thickness direction of the piezoelectric body so as to increase the capacity of the piezoelectric element, thereby improving the performance of the ultrasonic probe. Various inventions on the two-dimensional array ultrasonic probe including such a laminated piezoelectric element are being proposed.

For example, JP-A 2000-138400 (KOKAI) discloses that a plurality of electrodes are selectively patterned so as to expose each electrode on one side surface of the laminated piezoelectric element, thereby making it possible to withdraw the signal line and the ground line, and that a signal line and a ground line formed on a flexible printed wiring board are connected to the signal line and the ground line exposed on the side surface of the laminated piezoelectric element, respectively, thereby forming an array group forming a single column.

Also, JP-A 2005-210245 (KOKAI) discloses that a two-dimensional array ultrasonic probe is realized by arranging the plurality of flexible printed wiring boards having laminated piezoelectric elements forming a single column arranged therein into the row direction so that the plurality of piezoelectric elements are arranged in matrix form.

The laminated piezoelectric element is constructed such that a plurality of electrodes are alternately arranged within a piezoelectric body in the thickness direction of the piezoelectric body, wherein the side edges alone of the first electrodes are exposed on one of the two mutually facing side surfaces of the piezoelectric body and the side edges alone of the second electrodes are exposed on the other side surface of the piezoelectric body, and voltage is applied between the first electrode and the second electrode. Therefore, the mounting method disclosed in JP-A 2005-210245 pointed out above is useful in handling a large number of fine laminated piezoelectric elements.

However, in the two-dimensional array ultrasonic probe having the laminated piezoelectric element incorporated therein, one side surface of the laminated piezoelectric element is connected to the printed wiring board, with the result that it is difficult to obtain symmetric directivity characteristics of the ultrasonic wave. To be more specific, since a printed wiring board with a large acoustic load is connected to the side surface of the laminated piezoelectric element, the vibrations of the laminated piezoelectric elements are rendered nonuniform so as to give rise to the asymmetric properties. If the directivity characteristics of the ultrasonic wave become asymmetric, the intensity of the echo reflected from the object is varied depending on the position of the object so as to lower the image quality in forming the image of the reflected echo.

One of the methods for dealing with the problem noted above is, for example, to connect the printed wiring board to both side surfaces of the laminated piezoelectric element so as to obtain symmetric directivity characteristics of the ultrasonic wave. However, the printed wiring board has a large acoustic load, as pointed out above. As a result, the angle of directivity becomes narrow and, thus, each of the transmitting-receiving region of the ultrasonic wave and the displayed image region is limited.

It should also be noted that it is possible to lower the influence given by the acoustic load by decreasing the thickness of the printed wiring board. However, if the thickness of the printed wiring board is decreased, the warping of the printed wiring board increases so as to make it difficult to arrange the laminated piezoelectric elements with a high accuracy. It follows that the resolution of the image is lowered.

As described above, it was difficult to arrange the laminated piezoelectric elements with a fine pitch at a high accuracy so as to make it difficult to obtain a two-dimensional array ultrasonic probe having excellent directivity characteristics of the ultrasonic wave in the past.

### BRIEF SUMMARY OF THE INVENTION

According to the present invention, there is provided a two-dimensional array ultrasonic probe, comprising:

a plurality of channels arranged with spaces in a two-dimensional direction, each channel including a laminated piezoelectric element and an acoustic matching layers formed on the laminated piezoelectric element, the laminated piezoelectric element including a plurality of first and second electrodes arranged alternately within a piezoelectric body in a thickness direction of the piezoelectric body such that the side edges alone of the first electrodes are exposed to one of the two mutually facing side surfaces of the piezoelectric body and the side edges alone of the second electrodes are exposed to the other side surface of the piezoelectric body;

a backing member having the laminated piezoelectric element of each channel mounted thereon;

a signal side electrode formed to extend from one side surface of the piezoelectric body included in the laminated piezoelectric element of each channel to reach the backing member, and connected to the side edges of the plural first electrodes exposed to the one side surface of the piezoelectric body;

a ground side electrode extending from the other side surface of the piezoelectric body included in the laminated piezoelectric element of each channel to reach the backing member and connected to the side edges of the plural second electrodes exposed to the other side surface of the piezoelectric body;

a signal side printed wiring board connected to the signal side electrode at the portion positioned in the backing member;

a ground side printed wiring board connected to the ground side electrode in the portion positioned in the backing member; and

a filling member charged in at least the spaces between the adjacent channels.

### BRIEF DESCRIPTION OF THE SEVERAL VIEWS OF THE DRAWING

FIG. 1 is an oblique view showing the construction of a two-dimensional array ultrasonic probe according to one embodiment;

FIG. 2 is a cross-sectional view along the line II-II shown in FIG. 1;

FIG. 3 is a cross-sectional view along the line III-III shown in FIG. 1;

FIG. 4 shows the construction of first and second electrodes formed in the laminated piezoelectric element included in the channel arranged on a backing member;

FIG. 5 shows anther construction of the first and second electrodes formed in the laminated piezoelectric element included in the channel arranged on a backing member; and

FIGS. 6A, 6B, 6C, 6D, 6E and 6F collectively show the manufacturing method of the two-dimensional array ultrasonic probe according to the embodiment.

### DETAILED DESCRIPTION OF THE INVENTION

A two-dimensional array ultrasonic probe according to one embodiment of the present invention will now be described with reference to the accompanying drawings.

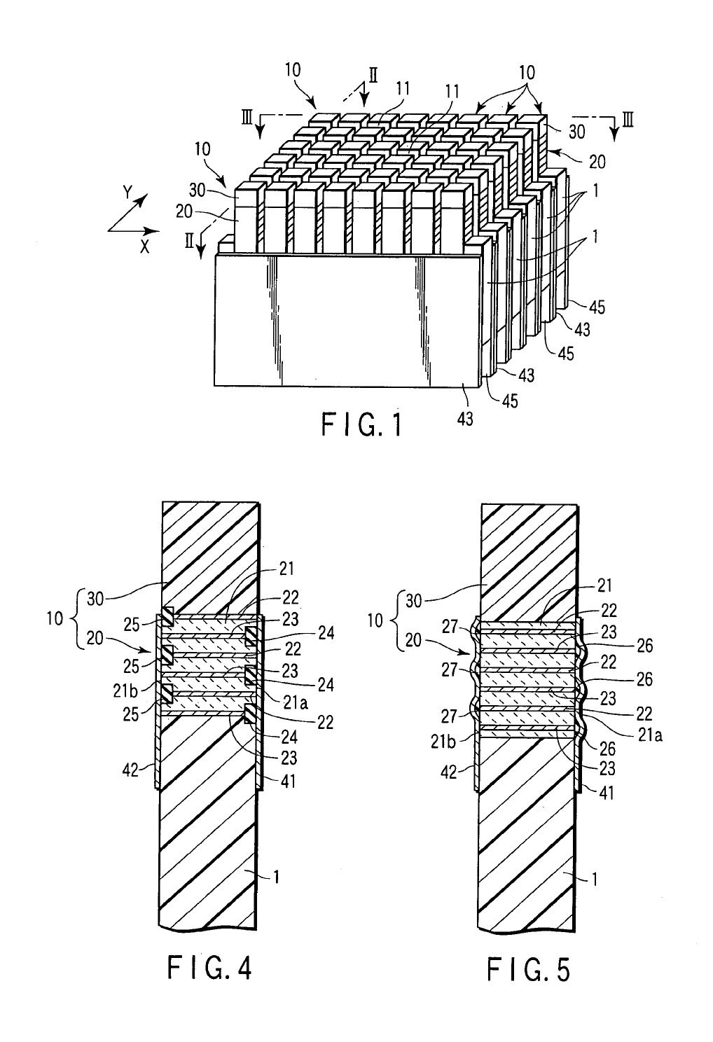

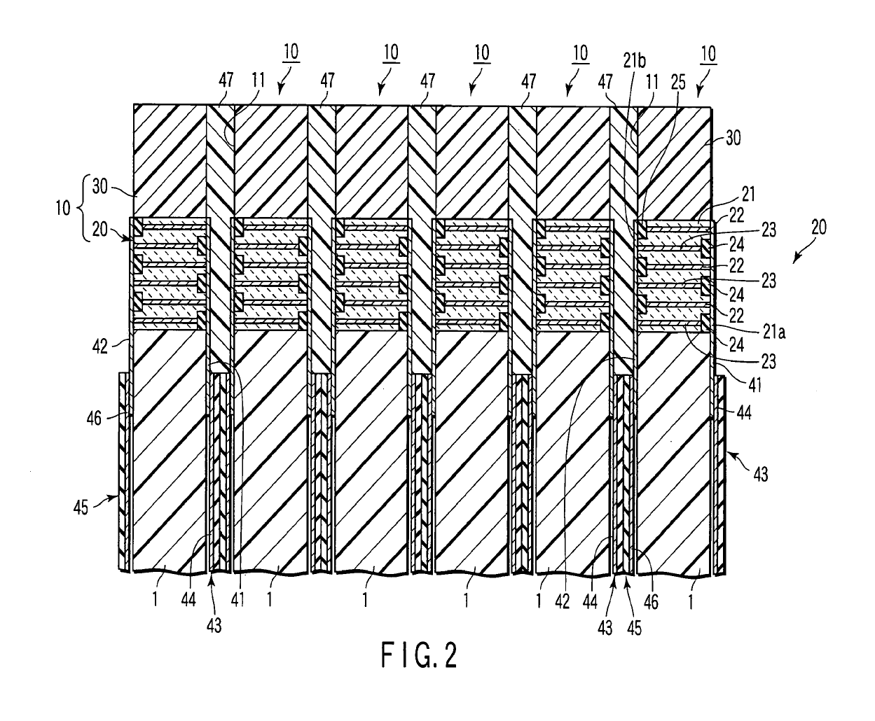

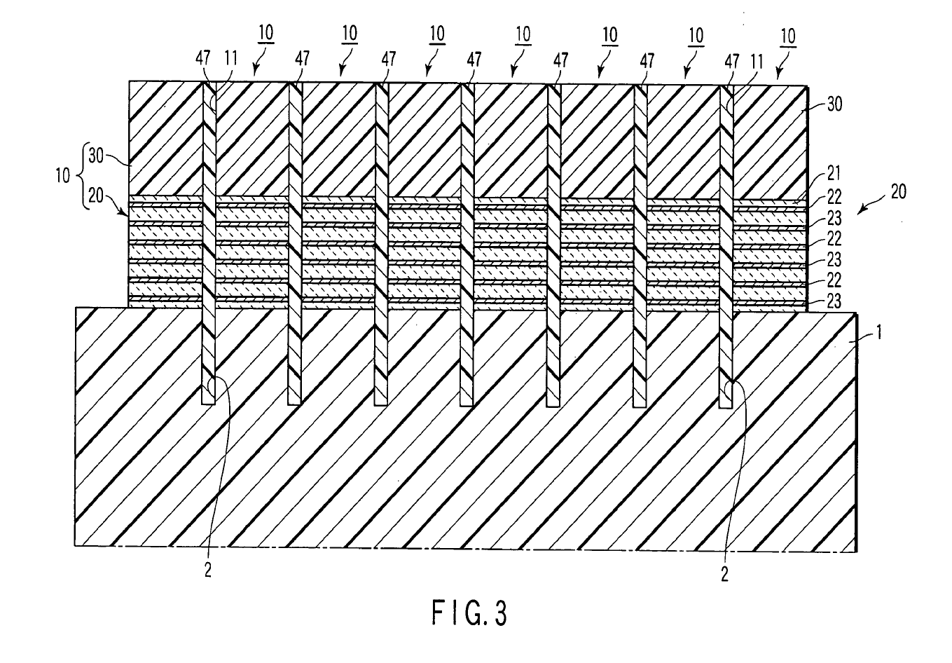

FIG. 1 is an oblique view showing the construction of a two-dimensional array ultrasonic probe according to one embodiment, FIG. 2 is a cross-sectional view along the line II-III shown in FIG. 1, and FIG. 3 is a cross-sectional view along the line III-III shown in FIG. 1.

The two-dimensional array ultrasonic probe comprises a plurality of strip-like backing members 1 extending in the X-direction. As shown in FIG. 1, the strip-like backing members 1 extending in the X-direction are arranged a prescribed distance apart from each other in the Y-direction. A plurality of channels 10 are arranged in a manner to form a matrix in the XY two-dimensional direction on the plural backing members 1 with spaces 11 formed between the adjacent channels 10. To be more specific, the plural adjacent channels 10 are arranged in the X-direction on a common backing member 1 as shown in FIG. 3. Also, the plural channels 10 are arranged in the Y-direction on a plurality of backing members 1, as shown in FIG. 2B. A plurality of trenches 2 are formed in each backing member 1 in a manner to correspond to the spaces 11 between the adjacent channels 10 arranged in the X-direction, as shown in FIG. 3. Incidentally, the backing member 1 serves to mechanically support a laminated piezoelectric element, which is described herein later, of each channel and to control the laminated piezoelectric element so as to shorten the ultrasonic pulse.

Each channel 10 comprises a laminated piezoelectric element 20 and an acoustic matching layer 30 of, for example, a single layer structure, which is arranged on the laminated piezoelectric element 20. It is possible for the acoustic matching layer 30 to be formed of a laminated structure consisting of at least two layers.

The laminated piezoelectric element 20 is arranged on each of the backing members 1 and is constructed such that a plurality of electrodes, e.g., six electrodes consisting of three first electrodes 22 and three second electrodes 23, are laminated one upon the other within a piezoelectric body 21 having a rectangular cross section. The first and second electrodes noted above are laminated one upon the other in the thickness direction of the laminated piezoelectric element 20. The piezoelectric body 21 is formed of, for example, a lead zirconate titanate (PZT) series piezoelectric ceramic material or a relaxer series single crystalline material. Each of the first and second electrodes 22 and 23 is formed of, for example, a Pd—Ag alloy. The four side surfaces of the piezoelectric body 21 having a rectangular cross section include two side surfaces 21a and 21b positioned to face each other in the Y-direction of each channel 10, i.e., in the arranging direction of the channel 10. On side edge alone of each of the first electrodes 22 is exposed to the side surface 21a noted above, and a side edge alone of each of the second electrodes 23 is exposed to the other side surface 21b noted above. To be more specific, an insulating member 24 formed of, for example, an epoxy resin is arranged on the side edge of each of the second electrodes 23, the side edge being positioned on the one side surface 21a of the piezoelectric body 2121, so as to cover the side edge of the second electrode 23. Also, an insulating member 25 formed of, for example, an epoxy resin is arranged on the side edge of each of the first electrodes 22, the side edge being positioned on the other side surface 21b of the piezoelectric body 21, so as to cover the side edge of the first electrode 22. A notch is formed in that portion of the piezoelectric body 21 including the side edge of each of the second electrode 23 which is positioned in one side surface 21a of the piezoelectric body 21, and the notch thus formed is filled with, for example, an epoxy resin so as to form the insulating member 24 in a manner to cover the side edge of each of the second electrodes 23 on said one side surface 21a. Likewise, a notch is formed in that portion of the piezoelectric body 21 including the side edge of each of the first electrode 22 which is positioned in the other side surface 21b of the piezoelectric body 21, and the notch thus formed is filled with, for example, an epoxy resin so as to form the insulating member 25 in a manner to cover the side edge of each of the first electrodes 23 on the other side surface 21b. Because of the particular construction, the side edge alone of each of the first electrodes 22 is allowed to be exposed to one side surface 21a of the piezoelectric body 21 and the side edge alone of each of the second electrodes 23 is allowed to be exposed to the other side surface 21b of the piezoelectric body 21.

As shown in FIG. 2, a signal side electrode 41 is formed to extend from one side surface 21a of the laminated piezoelectric element 20 so as to reach the backing member 1 and is connected to the side edge of each of the first electrodes 22 exposed to the one side surface 21a of the laminated piezoelectric element 20. Also, a ground side electrode 42 is formed to extend from the other surface 21b of the laminated piezoelectric element 20 so as to reach the backing member 1 and is connected to the side edge of each of a plurality of second electrodes 23 exposed to the other side surface 21b of the laminated piezoelectric element 20.

A signal side printed wiring boar 43, i.e., a flexible printed wiring board for signals, includes signal lines 44 that are patterned at the arranging pitch of the channels 10 on the surface. The signal lines 44 are electrically connected to each other at the portions where the signal side electrode 41 is connected to the backing member 1 of the signal side electrode 41. Likewise, the ground side printed wiring boar 45, i.e., a flexible printed wiring board for the ground, include ground lines 46 that are patterned at the arranging pitch of the channels 10 on the surface. The ground lines 46 are electrically connected to each other at the portions where the ground side electrode 42 is connected to the backing member 1 of the ground side electrode 42. Incidentally, it is possible to use a ground electrode plate that is not patterned as a common ground line. The signal side electrode 41 and the signal side printed wiring board 43 are connected to each other in the space 11 positioned between the adjacent backing members 1 in the Y direction shown in FIG. 2. Likewise, the ground side electrode 42 and the ground side printed wiring board 45 are connected to each other in the space 11 positioned between the adjacent backing members 1 in the Y direction shown in FIG. 2.

A filling member 47 is loaded in the space 11 between the adjacent channels 10 arranged in the X-direction, in trenches 2 of the backing member 1 communicating with the space 11 noted above, in the space 11 between the adjacent channels 10 arranged in the Y-direction, and is further loaded between the adjacent backing members 1.

An acoustic lens (not shown) is formed on the acoustic matching layer 30 included in each of the plural channels 10. The plural backing members 1, the plural channels 10 and the plural acoustic lenses (not shown) are housed in a case (not shown). A signal processing circuit (not shown) including a control circuit for controlling the drive timing of the laminated piezoelectric element 20 for each channel and an amplifying circuit for amplifying the received signal received by the laminated piezoelectric element 20 is housed in the case housing the plural backing members 1, the plural channels 10 and the plural acoustic lenses (not shown). A signal line 44 and a ground line 46 of the flexible printed wiring boards 43 and 45 are electrically connected to the control circuit and the amplifying circuit noted above.

It is desirable for the backing member 1 to be formed of a composite material obtained by incorporating a glass unwoven fabric into an epoxy resin. The backing member formed of the particular material makes it possible to improve the positioning accuracy of the plural channels 10 supported by the backing member 1. It is also possible to suppress the generation of the chipping or cracking of the laminated piezoelectric element 20 constituting the channel 10. It is desirable for the backing member 1 to have a sufficient thickness relative to the wavelength of the ultrasonic wave of a prescribed frequency used, i.e., to have a thickness adapted for sufficiently attenuating the ultrasonic wave in order to maintain the satisfactory acoustic characteristics exhibited by the two-dimensional array ultrasonic probe.

In the embodiment described above, three first electrodes 22 and three second electrodes 23 totaling 6 electrodes are alternately arranged within the piezoelectric body 21 in the thickness direction of the piezoelectric body 21 so as to form the laminated piezoelectric element 20 constituting each of the channels 10. However, the construction of the laminated piezoelectric element 20 is not limited to the example given above. For example, it is also possible for each of the first electrodes 22 and the second electrodes 23 to be formed of two electrodes or four or more electrodes which are arranged within the piezoelectric body 21.

The arranging mode of the first and second electrodes 22, 23 of the laminated piezoelectric element 20 and the mode of exposing the first and second electrodes 22, 23 to the mutually facing surfaces of the piezoelectric body 21 are not limited to those shown in FIG. 2.

Concerning the arranging mode of the first and second electrodes 22 and 23, it is possible to arrange the uppermost first electrode 22 on the upper surface of the piezoelectric body 21 and to arrange the lowermost second electrode 23 on the lower surface of the piezoelectric body 21. In this case, a plurality of additional first electrodes 22 and a plurality of additional second electrodes 23 are arranged between the uppermost first electrode 22 and the lowermost second electrode 23 such that the first electrodes 22 and the second electrodes 23 are alternately laminated one upon the other within the piezoelectric body 21, thereby obtaining the laminated piezoelectric element 20 as shown in FIG. 4. Where the laminated piezoelectric element 20 is constructed such that the edge surface of the uppermost first electrode 22 is allowed to be exposed to one side surface 21a alone of the piezoelectric body 21, a notch is formed to extend upward from the piezoelectric body 21 including the side edge of the uppermost first electrode 22 positioned on the other side surface 21b of the piezoelectric body 21 into the acoustic matching layer 30 positioned on the piezoelectric body 21, followed by filling the notch thus formed with an insulating member 25 consisting of, for example, an epoxy resin. On the other hand, where the edge surface of the lowermost second electrode 23 is allowed to be exposed to other side surface 21b alone of the piezoelectric body 21, a notch is formed to extend downward from the piezoelectric body 21 including the side edge of the lowermost second electrode 23 positioned on one side surface 21a of the piezoelectric body 21 into that portion of the backing member 1 which is positioned below the piezoelectric body 21, followed by filling the notch thus formed with an insulating member 24 consisting of, for example, an epoxy resin.

The side edges of the first and second electrodes 22 and 23 can be exposed to the corresponding surfaces of the piezoelectric body 21 by, for example, the method shown in FIG. 5. To be more specific, an insulating layer 26 consisting of, for example, an epoxy resin is formed on the side edge of each of the second electrodes 23 positioned on one side surface 21a of the piezoelectric body 21 so as to permit the side edge alone of each of the first electrodes 22 to be exposed to one side surface 21a of the piezoelectric body 21. Also, an insulating layer 27 consisting of, for example, an epoxy resin is formed on the side edge of each of the first electrodes 22 positioned on the other side surface 21b of the piezoelectric body 21 so as to permit the side edge alone of each of the second electrodes 23 to be exposed to the other side surface 21b of the piezoelectric body 21.

It is desirable for the acoustic impedance of the acoustic matching layer 30 to be set at a value intermediate between the acoustic impedance of the piezoelectric body 21 and the acoustic impedance of the object so as to permit the ultrasonic wave to be transmitted smoothly. Where the acoustic matching layer 30 is formed of a plurality of layers, it is desirable for the acoustic impedance to be gradually decreased from the acoustic matching layer 30 on the side of the laminated piezoelectric element 20 toward the acoustic lens. It is also desirable for the acoustic impedance to be close to that of the object.

Each of the signal side electrode 41 and the ground side electrode 42 is formed of a laminated metal film of, for example, a Cr/Au (front side) structure. It is desirable for each of these electrodes to have a thickness of 100 nm to 2 μm. If each of these electrodes is thinner than 100 nm, it is possible for each of the signal side electrode 41 and the ground side electrode 42 to be broken by the vibration of the laminated piezoelectric element 20. On the other hand, if the thickness of each of these electrodes exceeds 2 μm, the acoustic load is increased in each of the signal side electrode 41 and the ground side electrode 42, with the result that the directivity angle is narrowed and the transmitting-receiving region of the ultrasonic wave is limited so as to limit the image region that is displayed.

The printed wiring boards 43 and 45 for the signal and for the ground are not limited to the flexible printed wiring boards used in the embodiment described above. It is also possible for the flexible printed wiring boards 43 and 45 to be replaced by rigid printed wiring boards each comprising a substrate formed of a composite material obtained by incorporating the glass unwoven fabric into an epoxy resin and a conductive layer (signal line, ground line) formed on the surface of the substrate and consisting of at least one metal selected from the group consisting of Au, Cr, Cu and Ni.

The loading member 47 is made of, for example, a silicone resin.

As described above, according to an embodiment, the signal side electrode 41 is allowed to extend from one side surface 21a of the laminated piezoelectric element 20 included in each of the channels 10 to reach the backing member 1 and is connected to the side edge of each of the plural first electrodes 22 exposed to the side surface 21a noted above. Like wise, the ground side electrode 42 is allowed to extend from the other side surface 21b of the laminated piezoelectric element 20 to reach the backing member and is connected to the side edge of each of the plural second electrodes 23 that are exposed to the side surface 21b. As a result, the signal side printed wiring board 43 and the ground side printed wiring board 45 are connected, respectively, to those portions of the signal side electrode 41 and the ground side electrode 42 which are positioned in the backing member 1. In other words, it is possible to suppress the acoustic load applied by the printed wiring boards 43, 45 to the laminated piezoelectric element 20. In the embodiment, the application of the acoustic load is suppressed by avoiding the conventional construction that the signal side printed wiring board and the ground side printed wiring board are connected directly to the laminated piezoelectric element. As a result, it is possible for the laminated piezoelectric element 20 to produce symmetric directivity characteristics. It is also possible to provide a two-dimensional array ultrasonic probe that permits transmitting an ultrasonic wave into a region of a wide angle so as to obtain an image having a high resolution.

Further, the signal side printed wiring board 43 and the ground side printed wiring board 45 are connected to the signal side electrode 41 and the ground side electrode 42, respectively, in the regions that are positioned in the backing member 1. The particular construction makes it possible to avoid the inconvenience that an undesired vibration is added to the piezoelectric vibration of the laminated piezoelectric element 20. As a result, it is possible to use a material having a high mechanical strength for forming the substrate of each of the printed wiring boards 43 and 45. It follows that, even where the thickness of each of the printed wiring boards 43 and 45 is decreased, it is possible to inhibit the warping of the printed wiring boards 43, 45, with the result that the channels 10 can be arranged at a high accuracy.

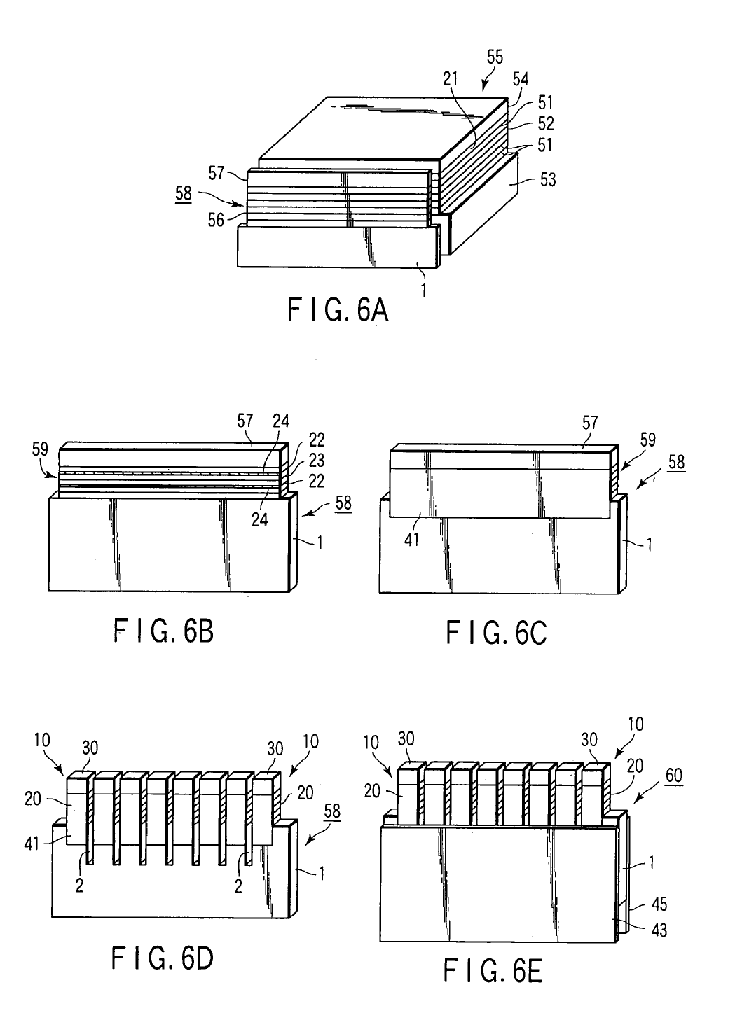

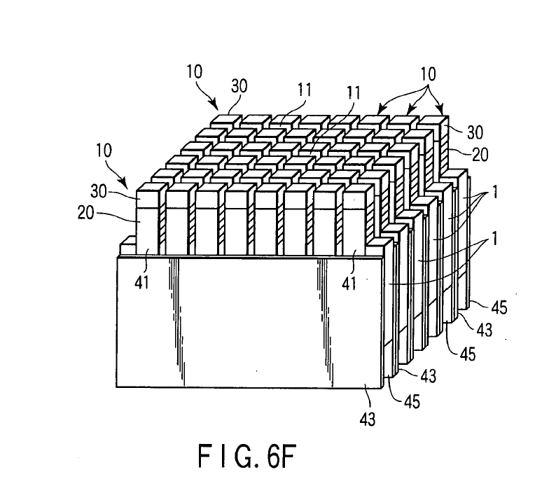

The method of manufacturing the two-dimensional array ultrasonic probe according to an embodiment will now be described in detail with reference to FIGS. 6A to 6F.

The method of manufacturing the two-dimensional array ultrasonic probe according to the embodiment of the present invention comprises (1) the step of preparing a strip-like laminated body including a backing member, a laminated piezoelectric element and an acoustic matching layer, (2) the step of connecting the strip-like laminated body to a printed wiring board so as to obtain a channel array unit of a single column arrangement, and (3) the step of laminating the channel array units of the single column arrangement in the row direction. Each step of the manufacturing method will now be described in detail.

1) Preparation of Strip-Like Laminated Body:

First, piezoelectric green sheets (piezoelectric body) each formed of, for example, lead zirconate titanate and having a thickness of 20 μm and electrode layers each formed of a Pd—Ag alloy and having a thickness of 2 μm are alternately laminated one upon the other, followed by sintering the laminate structure so as to obtain a plate-like sintered body 52 including a piezoelectric body 21 and inner electrodes 51 arranged within the piezoelectric body 21 in a manner to form six layers. The plate-like sintered body 52 thus obtained is bonded to a plate-like backing member 53 with an epoxy adhesive interposed therebetween. The plate-like backing member 53 can be prepared by mixing an oxide powder with, for example, a resin material or a rubber material. Particularly, it is desirable for the plate-like backing member 53 to be manufactured from a composite material prepared by incorporating a glass unwoven fabric into an epoxy resin. In the next step, a plate-like acoustic matching layer 54 formed of, for example, an epoxy resin that is processed in advance so as to have a prescribed acoustic impedance and a prescribed thickness is bonded to the upper surface of the plate-like sintered body 52 so as obtain a plate-like laminated body 55 consisting of the plate-like backing member 53, the plate-like sintered body 52 and the plate-like acoustic matching layer 54. Then, the plate-like laminated body 55 is cut at a width of, for example, about 400 μm by the dicing treatment so as to obtain a plurality of strip-like laminated bodies 58 each comprising the strip-like sintered body 56 in which the internal electrodes 51 forming 6 layers are alternately arranged on the piezoelectric body 21, and the strip-like acoustic matching layer 57, which are mounted in the order mentioned on the strip-like backing member 1, as shown in FIG. 6A. The width of the strip-like laminated body 58 thus cut out is set somewhat larger than the width of the channel that is finally required.

In the next step, a dicing treatment is applied to the piezoelectric body portion including the side edge of the electrode 51 exposed to one side surface that is positioned to face the other side surface in the longitudinal direction of the strip-like sintered body 56 of the strip-like laminated body 58. As shown in FIG. 6B, the dicing treatment is applied along the side edge of the piezoelectric body portion so as to form a groove in every two layers. Also, another dicing treatment is applied to the piezoelectric body portion including the side edge of the electrode 51 exposed to the other side surface. The dicing treatment is applied along the side edge of the electrode 51 so as to form a groove in every two layers such that the groove thus formed is deviated from that formed in said one side surface. Then, an epoxy adhesive is loaded in the groove formed in each of mutually facing side surfaces of the strip-like sintered body 56, followed by polishing the two side surfaces defining the groove. As a result, formed is the first electrode 22 arranged within the piezoelectric body, having the side edge exposed at one side surface, and having the side edge insulated at the other side surface with the insulating member 24. Also formed is the second electrode 23 arranged within the piezoelectric body, having the side edge exposed at the other surface, and the side edge insulated at one side surface with an insulating member (not shown). In this fashion, the strip-like laminated piezoelectric element 59 is formed on the backing member 1.

In the next step, the signal side electrode 41 is formed on the backing member 1 including one side surface having an insulating member 24 formed by the sputtering treatment as shown in FIG. 6C. Also the ground side electrode (not shown) is formed on the backing member 1 including the other side surface. In this case, the first electrode 22 is connected to the signal side electrode 41 alone and is insulated from the ground side electrode. On the other hand, the second electrode 23 is connected to the ground side electrode alone and is insulated from the signal side electrode 41. As a result, the signal side electrode 41 and the ground side electrode (not shown) are formed to extend from the side surface of the strip-like laminated piezoelectric element 59 to the inner region of the backing member 1. The region of the signal side electrode 41 extending to the backing member 1 is set shorter than the cutting distance (length) of the backing member 1 in the channel dividing process described herein later. Also, the region of the ground side electrode extending into the backing member 1 is set longer than the cutting distance (length) of the backing member 1 in the channel dividing process. Since the lengths of the signal side electrode 41 and the ground side electrode, which extend to the backing member 1, are set as described above, the signal side electrode 41 is divided by the channel dividing process for every channel. On the other hand, the ground side electrode is left to be electrically connected even after the channel dividing process. Incidentally, it is possible to form an electrode group divided for every channel by forming the ground side electrode by the method similar to the method of forming the signal side electrode 41. An electrode is formed on the backing member 1 by exposing the region close to the strip-like laminated piezoelectric element 59 by the masking.

In the manufacturing method of the strip-like laminated body described above, the plate-like sintered body 52 is bonded to the plate-like backing member 53. Also, the plate-like acoustic matching layer 54 is bonded to the upper surface of the plate-like sintered body 52. Further, a dicing treatment is applied to the plate-like laminated body 55 so as to take out the strip-like laminated body 58, followed by mutually connecting the internal electrodes of the strip-like sintered body 56. However, the method of manufacturing the strip-like laminated body is not limited to the process steps described above. For example, it is possible to prepare first a strip-like sintered body 56 including internal electrodes, followed by bonding a strip-like acoustic matching layer 57 and a strip-like backing member 1 to the upper and lower surfaces of the strip-like sintered body 56 so as to form a strip-like laminated body 58. Also, the method of forming the strip-like laminated piezoelectric element 59 is not limited to the method involving the step of forming an insulating groove in the piezoelectric body. For example, it is also possible to use the method of applying an epoxy series adhesive by the screen printing method to the electrodes on the two mutually facing side surfaces of the piezoelectric body so as to form insulating layers on the side surfaces of the piezoelectric body, as shown in FIG. 5.

2) Manufacture of Array Unit of Single Column Arrangement in which a Strip-Like Laminated Body is Connected to a Printed Wiring Board:

As shown in FIG. 6D, the strip-like laminated body 58 is cut by, for example, a dicing saw from the side of the strip-like acoustic matching layer 57 toward the strip-like backing member 1 so as to divide the strip-like acoustic matching layer 57 and the strip-like laminated piezoelectric element 59, thereby forming a plurality of channels 10 each including the laminated piezoelectric element 20 and the acoustic matching layer 30. In general, each channel 10 has a width of 100 to 300 μm. Also, since the backing member 1 is cut in a depth of about 100 to 300 μm so as to form trenches 2, the signal electrode 41 extending to reach one side surface of the backing member 1 is divided for every channel. It should be noted, however, that the ground side electrode (not shown) extending to reach the other side surface of the backing member 1 is left to be used as a electrically connected electrode even after the channel dividing process.

In the next step, a signal side flexible printed wiring board 43 having signal lines (not shown) having a thickness of, for example, 20 μm or less, which are patterned at the arranging pitch of the laminated piezoelectric element 20, are mounted on one side surface of the backing member 1 so as to be electrically connected to the divided signal side electrode 41. It is possible for the flexible printed wiring board 43 to be bonded by using an epoxy series adhesive or an adhesive prepared by mixing a metal filler with an epoxy series adhesive. Even in the case of using the epoxy series adhesive, the excess adhesive is pushed out by the compression bonding because a fine irregularity is formed on the electrode surfaces, with the result that the signal lines of the printed wiring board 43 and the signal side electrode 41 are electrically connected to each other. It is also possible to connect electrically the signal line of the printed wiring board 43 to the signal side electrode 41 by using a solder. Then, the ground side flexible printed wiring board 45 having the compatible ground lines (not shown) having a thickness of, for example, 20 μm or less is bonded to the other side surface of the backing member 1 so as to be connected to the ground side electrode (not shown). Specifically, the flexible printed wiring board 45 is bonded and connected to the compatible side surface electrode 5 on the opposite side of the strip-like backing member 1. The bonding of the ground side flexible printed wiring board 45 is performed by a method similar to the method of bonding the signal side flexible printed wiring board 43 described above.

By the process steps described above, the channels 10 for a single column consisting of the laminated piezoelectric element 20 and the acoustic matching layer 30 are arranged on the backing member 1 at a prescribed pitch so as to form a channel array unit 60 of a single column arrangement, in which the signal side electrode 41 and the ground side electrode (not shown) are connected to the signal line (not shown) and the ground line (not shown) on the flexible printed wiring boards 43, 45, respectively.

In the manufacturing process of the array unit described above, the strip-like laminated body 58 is bonded to the flexible printed wiring boards 43, 45 after the channel division. However, it is also possible to carry out the channel division after the flexible printed wiring boards 43, 45 are bonded to the strip-like laminated body 58. In this case, it is possible for the end portion of the signal line (not shown) of the signal side flexible printed wiring board 43, i.e., the terminal section that is connected to the signal side electrode 41, to be formed of the signal side electrode group divided at the arranging pitch of the laminated piezoelectric element 20, or, to be formed of the compatible electrode and divided in the step of the channel division of the strip-like laminated body 58 at the same time.

It is possible for the flexible printed wiring board noted above to be formed of a rigid printed wiring board comprising a substrate formed of a composite material obtained by incorporating a glass unwoven fabric in the epoxy resin and a conductive layer (signal line, ground line) formed on the surface of the substrate and consisting of at least one element selected from the group consisting of Au, Cr, Cu and Ni.

3) Process of Laminating the Channel Array Unit of a Single Column Arrangement in the Row Direction:

As shown in FIG. 6F, a two-dimensional array is prepared by stacking a plurality of channel array units 60 of a single column arrangement in the row direction, such that the signal side flexible printed wiring boards 43 on the side surface of the backing member 1 are allowed to abut against the ground side flexible printed wiring boards 45. In this case, the channel array units 60 are positioned and stacked side by side in the row direction so as to set the top surfaces of the acoustic matching layers 30 into the same plane.

In the next step, a filling member formed of, for example, a silicone resin (not shown) is charged in the spaces between the adjacent channels and trenches, and an acoustic lens is mounted to cover a plurality of channels, followed by putting the resultant structure in a case housing a control circuit for controlling the drive timing of the laminated piezoelectric element included in each channel and an amplifier circuit for amplifying the signal received by the laminated piezoelectric element so as to manufacture a two-dimensional array ultrasonic probe.

The manufacturing method according to the embodiment described above makes it possible to arrange at a fine pitch a plurality of channels 10 each having the laminated piezoelectric element 20 formed on the backing member 1 at a high accuracy, with the result that it is possible to manufacture a two-dimensional array ultrasonic probe exhibiting excellent directional characteristics of the ultrasonic wave.

In the manufacture of the two-dimensional array ultrasonic probe according to the embodiment described above, a channel array unit 60 of a single column arrangement is obtained by preparing a signal side flexible printed wiring board 43 having a signal line and a ground side flexible printed wiring board 45 having a ground line, followed by stacking the array units 60 in the row direction. Alternatively, it is also possible to use a single flexible printed wiring board having a signal line and a ground line formed on both surfaces in place of the flexible printed wiring board positioned between the adjacent array units 60. In the case of using the flexible printed wiring board noted above, it is possible to decrease the interval of the space between the adjacent array units 60. In other words, the pitch of the arrangement of the array units 60 can be decreased. As a result, the arranging pitch of the channels 10 can be narrowed so as to make it possible to improve the resolution of the two-dimensional array ultrasonic probe.

Further, in the embodiment described above, the backing member is formed of a composite material prepared by incorporating a glass unwoven fabric into an epoxy resin. Therefore, when a plate-like laminated body including the backing member is processed so as to obtain a strip-like laminated body, it is possible to prevent the occurrence of cracks or chipping in the plate-like sintered body. As a result, it is possible to suppress the unevenness between the channels in the transmitting-receiving sensitivity of the ultrasonic wave. It is also possible to process the plate-like laminated body into a thinner strip-like laminated body so as to make it possible to diminish the width of the space between the adjacent channels and, thus, the resolution of the image of the ultrasonic wave can be improved. Further, since the backing member permits holding the channel including the laminated piezoelectric element steadily, it is possible to prevent the positional deviation of the channel derived from the warping of the backing member.

In addition, as described previously, it is possible for the printed wiring board on the side of the signal line and on the side of the ground to be replaced by a rigid printed wiring board comprising a substrate formed of a composite material obtained by incorporating a glass unwoven fabric into an epoxy resin and a conductive layer (signal line, ground line) formed on the surface of the substrate and consisting of at least one element selected from the group consisting of Au, Cr, Cu and Ni. Since a sufficient strength can be maintained even if the conductive layer is formed thin on the surface of the substrate, the strip-like laminated body can be supported with a high stability after the printed wiring board is connected to the strip-like laminated body. As a result, it is possible to prevent the warp of the strip-like laminated body so as to prevent the positional deviation of the channel. In other words, it is possible to arrange the channels with a high accuracy.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A two-dimensional array ultrasonic probe, comprising:
a plurality of channels arranged with spaces in X and Y-directions, each channel including a laminated piezoelectric element and an acoustic matching layer formed on the laminated piezoelectric element, the laminated piezoelectric element including a piezoelectric body and a plurality of first and second electrodes arranged alternately within the piezoelectric body in a thickness direction of the piezoelectric body, such that the first electrodes are exposed to one of the two mutually facing side surfaces along the X-direction of the piezoelectric body and the second electrodes are exposed to the other side surface along the X-direction of the piezoelectric body;
a plurality of strip-like backing members extending in the X-direction arranged a prescribed distance apart from each other in the Y-direction and in which a plurality of trenches are formed in a manner to correspond to the spaces between adjacent channels arranged in the X-direction, a part of each strip-like backing member separated by each trench having on laminated piezoelectric element of each channel mounted thereon;
a signal side electrode formed to extend from one side surface of the piezoelectric body to one of the two mutually facing side surfaces along the X-direction of the backing member, and connected to the side edges of the plural first electrodes;
a ground side electrode formed to extend from the other side surface of the piezoelectric body to the other side surface along the X-direction of the backing member, and connected to the side edges of the plural second electrodes
a signal side printed wiring board connected to the signal side electrode at the portion positioned in the backing member;
a ground side printed wiring board connected to the ground side electrode at the portion positioned in the backing member; and
a filling member charged in at least the spaces between the adjacent channels.

2. The probe according to claim 1, wherein notches are formed in the piezoelectric body portions including the side edges of the second electrodes positioned on one side surface of the piezoelectric body and an insulating member is buried in these notches so as to cover the side edges of the second electrodes, and notches are formed in the piezoelectric body portions including the side edges of the first electrodes positioned on the other side surface of the piezoelectric body and an insulating member is buried in these notches so as to cover the side edges of the first electrodes, thereby allowing the side edges alone of the first electrodes to be exposed to one of the two side surfaces of the piezoelectric body and allowing the side edges alone of the second electrodes to be exposed to the other side surface of the piezoelectric body.

3. The probe according to claim 2, wherein the insulating member is formed of an epoxy resin.

4. The probe according to claim 1, wherein each of the signal side electrode and the ground side electrode is formed of a metal laminated film of Cr/Au (surface side) structure.

5. The probe according to claim 1, wherein each of the Signal side electrode and the ground side electrode has a thickness of 100 nm to 2 μm.

6. The probe according to claim 1, wherein the signal side printed wiring is formed of a rigid printed wiring board including an insulating substrate formed of a composite material prepared by incorporating a glass unwoven fabric into an epoxy resin and at least one conductive layer formed on the insulating substrate and consisting of at least one element selected from the group consisting of Au, Cr, Cu and Ni.

7. The probe according to claim 1, wherein the ground side printed wiring is formed of a rigid printed wiring board including an insulating substrate formed of a composite material prepared by incorporating a glass unwoven fabric into an epoxy resin and at least one conductive layer formed on the insulating substrate and consisting of at least one element selected from the group consisting of Au, Cr, Cu and Ni.

8. The probe according to claim 1, wherein the backing member is formed of a composite material prepared by incorporating a glass unwoven fabric into an epoxy resin.

9. The probe according to claim 1, wherein the filling member is made of a silicone resin.
